(19) United States
(12) Patent Application Publication
(10) Pub. No.: US 2006/0287506 A1
(43) Pub. Date: Dec. 21, 2006

(54) Method for separating and concentrating biological materials using continuous-flow ultracentrifugation

(75) Inventors: Marcus J. Horn, Parsippany, NJ (US); Donald K. McRorie, Sunnyvale, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Alfa Wassermann, Inc.

(21) Appl. No.: 11/153,282

(22) Filed: Jun. 15, 2005

(51) Int. Cl. C07H 21/04; C07K 16/18; C07K 14/775

(52) U.S. Cl. 530/359; 530/387.1; 530/412; 536/25.4

(57) ABSTRACT

The present invention is directed to a method of isolating and concentrating biological materials based on their buoyant density by subjecting a sample containing the biological materials to density-gradient ultracentrifugation. In one aspect of the present invention, a method for isolating at least one biological material is provided. A sample containing at least one biological material is introduced into an ultracentrifuge having a density-gradient established therein, and the sample is centrifuged until at least one biological material is isolated according to its buoyant density.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### BACKGROUND OF THE INVENTION

As the biological sciences have progressed, characterization of biological materials, such as biological molecules and organelles, has become increasingly important. Precise characterization of these materials opens the door to novel drug therapies for disease, as well as to a greater understanding of the mechanisms underlying many diseases. Ratios of high density to low density lipoproteins have been correlated to cardiovascular disease, with increasing attention being paid to various low concentration variants of each.

Many biological materials exhibit a buoyant density that can be used, among other things, to distinguish them from other, or similar materials. Such materials can be separated using density gradients and procedures such as, for example, differential centrifugation. For example, lipoproteins are composed of varying amounts of proteins and lipids. They differ not only by size and electrophoretic mobility, but also by buoyant density. Thus, in addition to other techniques available for separating, identifying, and classifying lipoproteins, density-gradient ultracentrifugation may be used.

Such methodologies have, however, had drawbacks, including the scale of the processes involved, as well as an inability to adequately detect and utilize fractions containing materials that are present only in dilute concentrations in the starting sample. What is needed, therefore, is a method for isolating biological materials that is scalable, that is, a method that can be utilized with smaller or larger volumes than those methods that currently exist in the art, and one that is capable of concentrating dilute materials so that increased information is obtained from sample analysis.

### BRIEF SUMMARY OF THE INVENTION

The present invention is directed to a method of separating, concentrating and accumulating biological materials based on their buoyant density by subjecting a sample containing the biological materials to continuous-flow density-gradient ultracentrifugation. In one aspect of the present invention, a method for isolating at least one biological material is provided. A sample containing at least one biological material is introduced into an ultracentrifuge having a density-gradient established therein, and the sample is centrifuged until at least one biological material is isolated according to its buoyant density.

In another aspect of the present invention, a volume of sample is provided, the volume exceeding the capacity of the ultracentrifuge rotor. A density-gradient is established within the ultracentrifuge rotor and the sample is continuously provided into the rotor while the rotor is spinning. As sample is entering the rotor, a like amount of fluid is removed from (or allowed to flow out of) the rotor. This process is continued until the entire sample has passed through the rotor and has been subjected to ultracentrifugation for a predetermined amount of time. The rotor is then allowed to come to a rest and the isolated sample is removed therefrom.

In another aspect of the present invention, the ultracentrifuge rotor provided has a capacity of from about 25 ml to about 8 L.

In another aspect of the present invention, the method described herein is used to isolate, separate, concentrate or accumulate a biological material having a buoyant density.

In another aspect of the present invention, the method described herein is used to isolate, separate, concentrate or accumulate a biological material, said biological material consisting of a first material, having a first buoyant density, bound to a second material, having a second buoyant density.

In yet another aspect of the present invention, the method described herein is used to isolate, separate, concentrate or accumulate a lipoprotein.

In still a further aspect of the present invention, the method described herein is used to isolate, separate, concentrate or accumulate a protein bound to a lipid.

In still a further aspect of the present invention, the method described herein is used to isolate, separate, concentrate or accumulate a lipid bound to a protein.

In still a further aspect of the present invention, the method described herein is used to isolate, separate, concentrate or accumulate a material bound to a lipid.

In still a further aspect of the present invention, the method described herein is used to isolate, separate, concentrate or accumulate a biological organism.

### BRIEF DESCRIPTION OF THE DRAWINGS

FIG. 1 is a schematic view of various steps involved in one aspect of the present invention.

### DETAILED DESCRIPTION OF THE INVENTION

Definitions

Unless otherwise defined, all technical terms used herein have the same meaning as commonly understood by one of ordinary skill in the art to which this invention belongs. To facilitate the understanding of the invention, certain terms as used herein are defined below as follows:

Bind(s) or Bound To: As used herein, the terms “bind(s)” refers to an activity wherein one molecule recognizes and adheres to a particular second molecule in a sample, but does not substantially recognize or adhere to other structurally unrelated molecules in the sample. Generally, a first molecule that “specifically binds” to a second molecule has a binding affinity greater than about 10⁵ to about 10⁶ molesaiter for that second molecule. “Bound to” is used to describe the relationship between two or more molecules that “bind” to one another as defined above. Further, the above terms include the use of covalent bonds.

Biological Material: As used herein, the term “biological material” includes non-living biological molecules such as lipoproteins, nucleic acids or nucleic acid molecules, including RNA, DNA, ribozymes, siRNA, and other nucleic acids, proteins, polypeptides, amino-acids, including modified proteins or polypeptides, antibodies, antibody fragments, receptors, and other proteins or polypeptides, as well as other organic molecules suitable for separation by the present method. As used herein, the term “biological material” does not include organisms such as viruses, bacteria, eukaryotes, and the like.

Biological Organism: As used herein, the term “biological organism” includes bacteria, bacterial spores, eukaryotic organisms, archaebacteria, fungi, trypanosomes, parasites, and various other biological organisms suitable for separation by the present method. As used herein, the term “biological organism” does not include viruses.

Separation: As used herein, the term “separation” includes separation, isolation, concentration, and accumulation of the material being separated. The terms “separation” and “separated” are to be interpreted broadly within the context of the present invention.

The present invention is directed to a method of separating biological materials or organisms based on their buoyant density by subjecting a sample containing the biological materials to density-gradient ultracentrifugation. The present invention provides a method operable at continuously variable volumes when compared to conventional techniques, and is also a contnuous-flow method of ultracentrifugation, in which samples having volumes greater than or less than the capacity of the rotor can be used. As the entire sample passes through the rotor, the biological materials being isolated and separated are also concentrated and accumulated, because the final volume in which the materials are isolated is less than the volume of the starting sample.

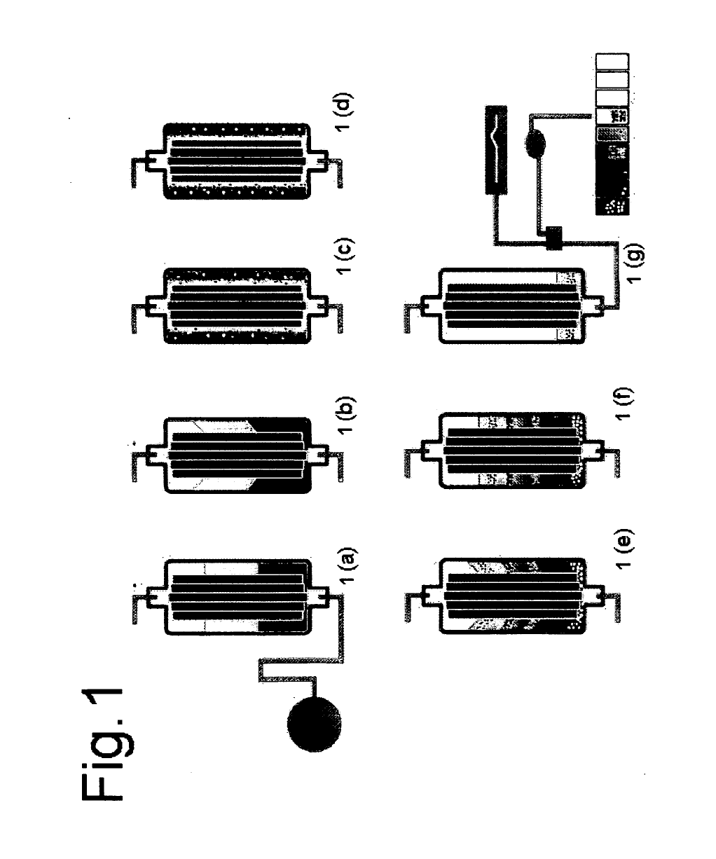

A schematic illustration of the present method is presented in FIG. 1. In FIG. 1(a) the loading of the gradient-forming solution is performed with the centrifuge rotor at rest. The gradient-forming solution is loaded via an inlet in the bottom of the centrifuge rotor. The various bands of density established in the gradient are illustrated by the white, grey, and black bands within the centrifuge rotor. FIG. 1(b) shows the reorientation of the established gradient during acceleration. The gradient begins to reform from a horizontal gradient to a vertical gradient. In other words, the gradient shifts from one established along a cross-sectional diameter of the centrifuge rotor to one established along a vertical length of the centrifuge rotor. This shifting of the gradient is due to centrifugal forces within the centrifuge rotor. FIG. 1(c) illustrates the introduction of a fluid sample (represented by black, white, and grey dots within the centrifuge rotor) into the centrifuge rotor. The fluid is preferably introduced via the fluid inlet at the bottom of the centrifuge rotor. The fluid sample flow indicated in FIG. 1(c) is allowed to continue until the entire sample from which components are to be isolated has passed through the centrifuge rotor and has spent sufficient time within the centrifuge rotor to be separated along the gradient established therein. Alternatively, in uses of the centrifuge rotor wherein dilute components present in the sample are to be concentrated, the continuous flow of fluid sample into the centrifuge rotor is allowed to continue until it is determined that a desired level of concentration has been reached.

FIG. 1(d) illustrates the condition of the sample and the established gradient once fluid sample flow into the centrifuge rotor has ended. As shown in the Figure, isopycnic banding of the separated sample is achieved. FIG. 1(e) illustrates another shifting of the density gradient during deceleration of the centrifuge rotor. As the density gradient shifts, the components of the separated sample remain in the density bands into which they were separated during operation of the centrifuge rotor. In FIG. 1(f), the centrifuge rotor is at rest and the shifting of the density gradient is complete. The density gradient has shifted from a vertical gradient back to a horizontal gradient, with each of the components of the sample remaining in the density band into which it was separated during operation of the centrifuge rotor. As shown in FIG. 1(g), removal of the sample is simple once the centrifuge rotor is at rest. The sample is removed back through the fluid inlet at the bottom of the centrifuge rotor and, because of the density gradient established within the centrifuge rotor, the sample is removed in discrete bands containing certain fractions with the biological materials being isolated from the sample. These fractions can be separated into receptacles such that various fractions contain the desired separated components. Thus, components of the sample have been effectively separated for analysis. In other uses of the present method, a dilute component of a sample is concentrated in a particular band in the density gradient and is removed in the same manner as that shown in FIG. 1.

Depending on the biological materials to be separated, various solutions, buffers, and operational parameters (such as centrifuge speed and time) must be used. Provided below are Examples detailing specific methodologies for isolating specific types of biological materials.

Various uses of the present invention will be apparent to those of skill in the art upon reading this disclosure. By way of example only, the following uses are detailed.

Antibodies

It is contemplated that the present method can be used to separate antibodies from a sample having antibodies contained therein. Various antibody types can be separated based upon their buoyant density. For example, IgG antibodies may be separated from IgM antibodies based on the differing buoyant densities of the two. Further, antibodies having specificity for certain molecules can be separated, either by differing buoyant densities of the antibodies themselves (due to differing structures in the variable regions or elsewhere) or by conjugating said antibodies with their target and separating them from the sample based on the buoyant density of the complex.

For example, if antibodies specific to a known compound A are sought to be separated from a sample (if present therein), compound A may be introduced into the sample and the resulting antibody:compound A complexes separated based on buoyant density.

Alternatively, if compound A is sought to be separated from a sample (if present therein), antibodies to compound A may be introduced into the sample and the resulting antibody:compound A complexes separated based on buoyant density.

Peptide Synthesis

The present method may be used to separate successfully-synthesized peptides from unsuccessfully-synthesized peptides based on differing buoyant densities of the two. For example, the initial portion of a peptide chain to be extended may be bound to a lipid or other support having buoyant density. Peptide synthesis is then performed on the initial portion of the peptide chain. Some desired peptides will be successfully synthesized, but others will fail for a variety of reasons. Once extension of the peptide chain is complete, the desired peptide, bound to a lipid or other support, will have a buoyant density that differs from that of undesirable peptides. The present method allows separation of these peptides based on that buoyant density.

Bound Materials

The present method may also be used to separate biological materials not generally having a buoyant density of their own. Such materials may be bound to a support, such as, for example, a polystyrene bead or a lipid, and separated based on the buoyant density of the desired material and bound support. In some cases, a desired material may be present in a sample but it may not be feasible to attempt to bind the desired material directly to a support for purposes of separation. Under such circumstances, a material with affinity for the desired material may be bound to a support and introduced into the sample. As the introduced material and support bind to the desired material, the desired material can then be separated based on the buoyant density of the complex.

Without further elaboration, it is believed that one skilled in the art can, using the preceding description, utilize the present invention to its fullest extent. The following specific example is offered by way of illustration and not by way of limiting this disclosure.

### EXAMPLE 1

### Isolation of Lipoproteins from Calf Plasma Using Continuous-Flow Ultracentrifugation

Sterile Bovine Plasma in 0.05% EDTA was obtained from Rockland, Inc. (Gilbertsville, Pa.). Three lots of serum were used, the three lots being obtained from three bleeds of two female calves, 12 to 17 months in age. Sample sizes of 150 mL, 500 mL, and 1 L were used in the present example for comparative purposes.

Continuous-flow density-gradient ultracentrifugation was performed using an Alfa Wassermann PKII centrifuge with an 800 mL rotor core. The rotor was initially filled with 0.05% EDTA. After clearing air from all channels, 400 mL of 60% w/v sucrose/0.05% EDTA was pumped into the bottom of the rotor with the rotor being at rest. Ramped acceleration was used to establish a linear 0-60% gradient, minimizing the mixing of sucrose during the acceleration process. Sample was loaded with the rotor running at 30K rpm, and after loading the rotor was run at 40K rpm for four hours. The rotor was then brought to rest using a controlled deceleration (to again minimize mixing), and 15 mL sample fractions were collected.

Table 1 shows the protein recovery for the 500 mL sample. The sample was loaded onto the continuous-flow ultracentrifuge, followed by a 150 mL buffer rinse. The wash-through was collected. The protein assay for of the collected fractions, wash-through, and original sample were all conducted under the same conditions.

TABLE 1
Continuous-flow ultracentrifuge protein recovery

| | Total Protein | % of Original Sample |
|---|---|---|
| Original Sample | 56,700 mg | 100% |
| Wash-Through | 15,400 mg | 27% |
| Collected Fractions | 40,200 mg | 71% |

The protein concentration of each fraction was measured according to the Bradford protein assay, which is known to those of skill in the art.

A Bio-Rad Criterion precast gel system (Bio-Rad Laboratories; Hercules, Calif.) with 4-15% resolving gels with 26 wells and Tris-Glycine buffer were used for non-denaturing gel electrophoresis. Ten microliters of protein from selected fractions were applied to each of the wells in the gel. The gels were run at a constant amperage of 20 mA per gel. After electrophoresis, the gels were stained with 0.03% Sudan Black in 30% methanol/30% isopropanol for 30 minutes, followed by destaining with 30% isopropanol.

After electrophoresis, gel bands stained with Sudan Black were sliced from the gel after soaking in water overnight. The gels were treated with N-Glyconase (Peptide N-Glycosidase F, Prozyme, San Leandro, Calif.) overnight at 37° C., without denaturants or detergents, to remove the N-glycosylated carbohydrates that are associated with most lipoproteins. The slices were reduced with TCEP (Tris (2-carboxyethyl) phosphine hydrochloride) and alkylated with IAM (iodoacetamide) before digestion with 0.02 mg/mL trypsin overnight at 37° C. Peptide samples were analyzed on a Thermo Electron LCQ Deca XP with NSI source.

The samples were eluted using a linear gradient of 5% solvent B (0.1% formic acid in acetonitrile) to 65% solvent B for 30 minutes with a flow rate of approximately 200 numinute. Mass spectrometry was conducted in a data-dependent MS/MS mode using a normalized collision energy of 35%. The capacity of the temperature of the ion source was set at 180° C. The resulting mass spectra data were searched against the NCBI nonredundant protein database using SEQUEST.

The results of the present Example showed that continuous-flow ultracentrifugation is a scalable process. Further, it was shown that increased sample loads leads to improved protein identification. Table 2, below, summarizes the findings:

TABLE 2
Continuous-flow ultracentrifugation is scalable; increasing
sample loads improves protein identification

| | | # Peptide Identified | |
|---|---|---|---|
| | Lipoprotein | 500 mL | 1000 mL |
| Heavy | A chain A, Receptor binding domain of | 2 | 7 |
| | a-2-macroglobulin | | |
| | FGBOB fibrinogen beta chain | 2 | 3 |
| HDL | C chain C, The crystal structure of | 2 | 2 |
| | modified bovine fibrinogen | | |
| | A chain A, The crystal structure of | 3 | 4 |
| | modified bovine fibrinogen | | |
| Light | Apolipoprotein A-I | 15 | 20 |
| LDL | Apolipoprotein C-II | 0 | 3 |
| | Apolipoprotein A-II | 0 | 2 |
| | Serine (or cysteine) proteinase inhibitor | 3 | 10 |
| LDL | Apolipoprotein B-100 | 4 | 5 |
| | Immunoglobulin heavy chain constant | 0 | 5 |
| | region | | |

The method provided in the present example, as outlined above, resulted in the separation of lipoprotein particles according to their densities, as well as effective concentration of the lipoproteins as compared to their starting plasma samples. As sample size increased from 150 mL, to 500 mL, to 1000 mL, increasing amounts of information were obtained.

It will be obvious to those of skill in the art upon reading this disclosure that many variations of the present method are possible without departing from the spirit or scope of the invention described herein. The number and kind of modifications that may be made to the present method are varied and large, and it is contemplated that such modifications are within the scope of the present invention. The specific embodiments described herein are given by way of example only, and the present invention is limited only by the appended claims.

1. A method of separating at least one biological material comprising the steps of:
a) continuously introducing a sample containing said at least one biological material into an ultracentrifuge rotor having a density gradient established therein;
b) centrifuging said sample within said ultracentrifuge rotor until at least a portion of said at least one biological material in said sample is separated according to the density of said at least one biological material; and
c) removing said at least one biological material from said rotor.

2. A method according to claim 1 wherein said ultracentrifuge rotor has a capacity of from about 25 mL to about 8 L.

3. A method according to claim 1 wherein said biological material has a buoyant density.

4. A method according to claim 1 wherein said biological material is bound to a second material, said second material having a buoyant density.

5. A method according to claim 4 wherein said second material is selected from the group consisting of lipids and polystyrene beads.

6. A method according to either of claims 4 or 5 wherein said biological material is selected from the group consisting of lipoproteins, DNA, RNA, proteins, polypeptides, ribozymes, and antibodies.

7. A method according to claim 6 wherein said biological material is a lipoprotein.

8. A method according to claim 1 wherein said biological material is selected from the group consisting of lipoproteins, DNA, RNA, proteins, polypeptides, ribozymes, and antibodies.

9. A method according to claim 8 wherein said biological material is a lipoprotein.

10. A method according to claim 2 wherein said biological material has a buoyant density.

11. A method according to claim 2 wherein said biological material is bound to a second material, said second material having a buoyant density.

12. A method according to claim 11 wherein said second material is selected from the group consisting of lipids and polystyrene beads.

13. A method according to either of claims 11 or 12 wherein said biological material is selected from the group consisting of lipoproteins, DNA, RNA, proteins, polypeptides, ribozymes, and antibodies.

14. A method according to claim 13 wherein said biological material is a lipoprotein.

15. A method according to claim 2 wherein said biological material is a lipoprotein.

16. A method according to claim 2 wherein said biological material is selected from the group consisting of lipoproteins, DNA, RNA, proteins, polypeptides, ribozymes, and antibodies.

17. A method according to claim 16 wherein said biological material is a lipoprotein.

18. A method for separating at least one biological material comprising the steps of:
a) providing a volume of sample containing at least one biological material to be separated;
b) providing an ultracentrifuge rotor adapted to receive a volume of sample less than the total volume of sample provided in step a);
c) establishing a density gradient within said ultracentrifuge rotor;
d) introducing a second volume of said sample into said rotor, said second volume of said sample being no greater than the volume the ultracentrifuge rotor is adapted to receive, said second volume being introduced into said rotor while said rotor is spinning, thereby separating said at least one biological material contained within said sample according to the density of said at least one biological material;
e) continuously introducing said sample into said rotor while said rotor is spinning;
f) continuously removing a fluid from said rotor at a rate about the same as that at which sample is entering said rotor;
g) iteratively performing steps e) and f) until the entire volume of said sample has passed through said rotor; and
h) removing said at least one biological material from said rotor.

19. A method according to claim 18 wherein said ultracentrifuge rotor has a capacity of from about 25 mL to about 8 L.

20. A method according to claim 19 wherein said biological material has a buoyant density.

21. A method according to claim 19 wherein said biological material is bound to a second material, said second material having a buoyant density.

22. A method according to claim 21 wherein said second material is selected from the group consisting of lipids and polystyrene beads.

23. A method according to either of claims 21 or 22 wherein said biological material is selected from the group consisting of lipoproteins, DNA, RNA, proteins, polypeptides, ribozymes, and antibodies.

24. A method according to claim 23 wherein said biological material is a lipoprotein.

25. A method according to claim 19 wherein said biological material is selected from the group consisting of lipoproteins, DNA, RNA, proteins, polypeptides, ribozymes, and antibodies.

26. A method according to claim 25 wherein said biological material is a lipoprotein.

27. A method according to claim 19 wherein said biological material has a buoyant density.

28. A method according to claim 19 wherein said biological material is bound to a second material, said second material having a buoyant density.

29. A method according to claim 19 wherein said biological material is a lipoprotein.

30. A method of separating at least one biological organism comprising the steps of:
a) continuously introducing a sample containing said at least one biological organism into an ultracentrifuge rotor having a density gradient established therein;
b) centrifuging said sample within said ultracentrifuge rotor until at least a portion of said at least one biological organism in said sample is separated according to the density of said at least one biological organism; and
c) removing said at least one biological organism from said rotor.

31. A method according to claim 30 wherein said ultracentrifuge rotor has a capacity of from about 25 mL to about 8 L.

32. A method according to claim 30 wherein said biological organism has a buoyant density.
